(19) United States
(12) Patent Application Publication
(10) Pub. No.: US 2021/0230690 A1
(43) Pub. Date: Jul. 29, 2021

(54) BIOSENSOR

(71) Applicant: Katholieke Universiteit Leuven, Leuven (BE)

(72) Inventors: Jeroen Lammertyn, Neerijse (BE); Saba Safdar, Leuven (BE); Dragana Spasic, Kessel-Lo (BE)

(21) Appl. No.: 17/053,538

(22) PCT Filed: May 8, 2019
(86) PCT No.: PCT/EP2019/061799

§ 371 Date: Nov. 6, 2020

(30) Foreign Application Priority Data
May 8, 2018 (GB) 1807482.3
Jun. 11, 2018 (GB) 1809545.5
Jun. 11, 2018 (GB) 1809547.1
Jun. 11, 2018 (GB) 1809550.5
Jun. 13, 2018 (LU) 100825

(51) Int. Cl. C12Q 1/6876; C12N 9/22; C12N 15/11

(52) U.S. Cl. CPC C12Q 1/6876; C12N 9/22; C12N 2310/20; C12N 2800/80; C12N 15/11

(57) ABSTRACT

Present invention in general concerns (re-)engineering the CRISPR/CAS guide RNA for protein independent signal generation.

### BACKGROUND OF THE INVENTION

### A. Field of the Invention

Present invention in general concerns (re-)engineering the CRISPR/CAS guide RNA for protein independent signal generation. In a particular embodiment the present invention relates to a comprising CRISPR/Cas guide RNA with a tracrRNA-NAzyme hybrid and more particular engineering tracrRNA-NAzyme hybrid can be engineered to have a 10-23 NAzyme motif or a 8-17 core NAzyme motif for catalytic activity.

### B. Description of the Related Art

CRISPR/Cas is biological complex, comprising of nucleic acid component and protein component. The quintessential Streptococcus pyogenes SpCas9 complex includes a guide RNA (made up of CRISPR RNA (crRNA) and trans-activating CRISPR RNA (tracrRNA) parts) and an associated protein Cas9 (CRISPR associated protein 9). The CRISPR/Cas complex specifically binds with target nucleic acid, and cleaves it. The target nucleic acid is defined by the sequence of guide RNA, the site of cleavage is dependent upon both the guide RNA and the Cas, and Cas is responsible for the cleavage of target nucleic acid. A dead version of Cas, dCas, retains all sequence specificity and recognition activity of the original Cas, without the cleavage activity (see FIG. 1)

Both wildtype crRNA-tracrRNA and the single fusion guide RNA (sgRNA) have been successfully used for many applications, ranging from genome editing and DNA tagging, to diagnostics. In all these applications, the amplified signal is generated from cleavage activity of Cas protein, binding of dCas protein conjugated with another protein, or external signalling molecule separate from the CRISPR/Cas complex. Strategies utilizing the guide RNA for signal generation are non-catalytic and do not amplify the signal.

In present invention on the other hand the guide RNA component of a CRISPR system, for instance the CRISPR/Cas complex, is engineered by introducing a NAzyme sequence in tracrRNA sequence, resulting in a hybrid RNA-DNA molecule. In a CRISPR/Cas complex, the hybrid guide RNA successfully forms functional complex with Cas9 and dCas9 and the complex with hybrid successfully binds and cleaves target DNA. This way the engineered CRISPR/Cas complex comprising a guide RNA with NAzyme sequence comprised in the tracrRNA sequence can be used for amplified signal generation in conjunction with native CRISPR/Cas activities.

### SUMMARY OF THE INVENTION

The present invention solves the problems of the related art by engineering DNAzyme sequences into the guide RNA of a CRISPR/Cas systems. It was surprisingly found that the hybrid DNA-RNA form is still compatible with function of CRISPR/Cas system, while the NAzyme activity is still intact after complex formation. Although the NAzyme itself also has nucleic acid cleavage activity, and any cross-reactivity would undermine the efficacy of the system, it was demonstrated that the hybrid RNA does not interfere with the cleavage of Cas9.

In accordance with the purpose of the invention, as embodied and broadly described herein, the invention is broadly drawn to system of biosensing that is applicable to all CRISPR/Cas systems, regardless of which Cas protein.

In one aspect of the invention, concerning a method for producing a CRISPR/Cas complex based sensor, the method comprising engineering a chemically reactive nucleic acid (NAzyme) in the tracrRNA of the guide RNA component of the CRISPR.

Another aspect of the invention is engineered CRISPR comprising a tracrRNA-NAzyme hybrid.

Still another aspect of the invention, is a sensor kit, characterised in that the sensor kit comprises a hybrid tracrRNA-NAzyme in a complex with Cas-crRNA, with Cas9-crRNA, with dCas-crRNA, or with dCas9-crRNA and further comprising a labelled oligonucleotide substrate for NAzyme, preferably a fluorophore-labelled oligonucleotide substrate for NAzyme.

Yet another aspect of the invention is a method of analysing a selected DNA target, characterised in that the method comprises contacting said target DNA to be analysed with a tracrRNA-NAzyme Cas-crRNA complex or with a tracrRNA-NAzyme Cas9-crRNA complex or with tracrRNA-NAzyme dCas-crRNA complex or with tracrRNA-NAzyme dCas9-crRNA complex of any of the previous claims and adding a labelled NAzyme substrate for the said NAzyme in the said complex.

Present furthermore invention comprises the following aspect.

An object of present invention concerns a method of forming a sensor for characterising a target analyte, characterised in that the method comprises engineering a complex of a nucleic acid enzyme (NAzyme) in the tracrRNA of the guide RNA component of the CRISPR. In particular such bio sensor can be formed by engineering NAzyme in the tracrRNA of the guide RNA component of CRISPR/Cas and incubating the engineered NAzyme-tracrRNA hybrid with crRNA and Cas to form a complex.

Another object of present invention concerns a method of forming a sensor for characterising a target analyte by introducing a NAzyme in the tracrRNA of CRISPR and incubating said NAzyme-tracrRNA hybrid with crRNA and Cas9 or dCas9 to form a complex.

Yet another object of present invention concerns a method of forming a sensor for characterising a target analyte, characterised in that the method comprises engineering a complex of an engineered tracrRNA-NAzyme hybrid and introducing the hybrid in a CRISPR/Cas complex.

These above mentioned biosensors can be attached to a solid support such as a nano/microparticle or a component of a biosensor chip for instance in the reaction portion of that chip. Moreover these above mentioned biosensors can be attached to a support such as a nano/microparticle or a component of a capillary sensor analysis system for analyzing a sample liquid with respect to an analyte contained therein, in particular for analyzing a body fluid of humans or animals. In general such system comprising capillary sensors, including a capillary channel an inlet opening for the sample liquid and a vent opening, the capillary channel containing reagents, the reaction of the sample liquid with the reagents resulting in a measurable change of a measurement variable which is characteristic for the analysis, and an evaluation instrument.

These above mentioned sensors can be a biological component that combined with a physicochemical detector or that is combined with an optical detector. Or these above mentioned sensors can be a biological component that is attached to a physicochemical detector or that is attached to an optical detector.

A possible analyte to be analysed by interacting with the biological sensors of present invention can be characterised in that the analyte is a target polynucleotide, the target analyte is a target protein or peptide or the target analyte is of the group of polynucleotide-protein hybrid, polynucleotide-polypeptide hybrid; polynucleotide-oligopeptide hybrid and oligonucleotide-polypeptide hybrid.

Present furthermore invention comprises the following aspect.

A particular aspect of present invention is a biosensor comprising an engineered CRISPR system comprising a tracrRNA-NAzyme hybride and a transducer element associated with said CRISPR system or a biosensor comprising a transducer element associated with an engineered CRISPR system comprising a tracrRNA-NAzyme hybride. Suitable for this biosensor is a CRISPR system that is an engineered CRISPR/Cas comprising a tracrRNA-NAzyme hybrid comprised in a Cas-crRNA-tracrRNA complex or an engineered CRISPR/Cas comprising a tracrRNA-NAzyme hybrid comprised in a Cas9-crRNA-tracrRNA complex or in dCas-crRNA-tracrRNA complex or in dCas9-crRNA-tracrRNA complex. In the biosensor the transducer element can be of the group consisting of physicoelectrical transducer, a physicochemical transducer, and/or an optochemical transducer. This biosensor invention provides a way to characterise a target of the group consisting of a nucleic acid, a protein/peptide, lipid, polysaccharide, a cell surface, an oligonucleotide, a polynucleotide, an oligopeptide, a polypeptide, an oligonucleotide/oligopeptide hybrid, an oligonucleotide/ polypeptide hybrid, an oligonucleotide/oligopeptide hybrid and a polynucleotide/ polypeptide hybrid.

This biosensor invention can in one aspect transform the binding and catalytic effect of the CRISPR system into a detectable/measureable electrical indicator.

In a further embodiment of the invention, the biosensor of present invention comprises in a biosensor system further comprising a measuring instrument which measures biological information on an analyte supplied to the biosensor, wherein the biosensor comprises: a reaction portion which is formed to be connected to the transducer element.

In a particular embodiment of present invention the biosensor of present invention is comprised of a biosensor chip. This biochip can furthermore be connected with a measuring instrument which measures biological information on a biological material supplied to the biosensor chip. For instance the biosensor chip can be connected with a measuring instrument which measures biological information on a biological material supplied to the biosensor chip, wherein the biosensor chip comprises: a reaction portion with CRISPR system which reaction portion is formed to be electrically or optically connected to a plurality of sensor electrodes and to which the biological material is supplied, characterised in that what the reaction portion comprises.

In a particular embodiment of present invention the biosensor of present invention is comprised in a capillary sensor analysis system for analyzing a sample liquid with respect to an analyte contained therein, in particular for analyzing a body fluid of humans or animals, comprising capillary sensors, including a capillary channel an inlet opening for the sample liquid and a vent opening, the capillary channel containing reagents, the reaction of the sample liquid with the reagents resulting in a measurable change of a measurement variable which is characteristic for the analysis, and an evaluation instrument, which capillary sensor analysis system includes reagents that comprise said CRISPR system.

In another aspect, the present invention provides an electrochemical sensor for determining the concentration of an analyte in a sample, the sensor comprising an electrode, in use connected to external electronics of a measuring device and characterised in that it comprises a reaction zone with the CRISPR system of present invention as described here above.

Yet another aspect, the present invention is a sensor kit, characterised in that the sensor kit comprises CRISPR system of present invention as described here above. This sensor kit can be characterised in that it comprises an hybrid tracrRNA -NAzyme comprised in a complex with Cas-crRNA, with Cas9-crRNA or with dCas9-crRNA and further comprising a labeled oligonucleotide substrate for NAzyme or it can be characterised in that it comprises an hybrid tracrRNA -NAzyme comprised in a complex with Cas-crRNA, with Cas9-crRNA or with dCas-crRNA or with dCas9-crRNA and further comprising a substrate for NAzyme, such as fluorophore-labelled oligonucleotide. Any one of the previous embodiments the nucleic acid enzyme is of the group consisting of a DNAzyme, RNAzyme, DNAzyme-RNAzyme hybrid, a multi-component deoxyribozyme (MNAzyme), or any combination thereof.

In some embodiments, the invention provides a method for analyzing a target anaylte in a biological sample, comprising a) contacting an analysis ligand comprising an engineered tracrRNA-NAzyme hybrid CRISPR system with a biological sample for a time sufficient to form a target-analysis ligand complex, b) converting the complex reaction into a measurable signal and c) transducing signal into a readable result. Further suitable analysis ligand to execute the method of present invention are of the group of an engineered CRISPR system is a CRISPR/Cas comprising a tracrRNA-NAzyme hybrid comprised in a Cas-crRNA-tracrRNA complex, an engineered CRISPR system with an engineered CRISPR/Cas comprising a tracrRNA-NAzyme hybrid comprised in a Cas-crRNA-tracrRNA complex or in dCas-crRNA-tracrRNA complex or in a Cas9-crRNA-tracrRNA complex or in dCas9-crRNA-tracrRNA complex, an engineered CRISPR system with a NAzyme substrate, such as one which has a fluorophore on one end and a quencher on the other end so that the fluorescence increases when by cleavage the distance between the fluorophore and quencher increases and any of those whereby the catalytic motif of tracrRNA-NAzyme hybrid is a 10-23 NAzymes motif, and any of those whereby the catalytic motif is a 8-17 NAzyme motif. Any of these embodiments whereby the a NAzyme is of the group consisting of a DNAzyme, RNAzyme, DNAzyme-RNAzyme hybrid, a multi-component deoxyribozyme (MNAzyme), or any combination thereof.

Some of the methods described above may be embodied for analysing a selected DNA target, characterised in that the method comprises probing said target DNA to be analysed with a tracrRNA -NAzyme Cas-crRNA complex or with a tracrRNA-NAzyme Cas9-crRNA complex or with a tracrRNA-NAzyme dCas-crRNA complex or with a tracrRNA-NAzyme dCas9-crRNA complex of any of the previous embodiments and adding a labelled NAzyme substrate for the said NAzyme in the said complex. Some of the methods described above may be embodied for analysing a target analyte selected from the group consisting of RNA, cDNA, and genomic DNA, the cDNA being obtained from RNA by reverse transcription. Some of the methods described above may be embodied for analysing a target of the group consisting of a nucleic acid, a protein/peptide, lipid, polysaccharide, a cell surface, an oligonucleotide, a polynucleotide, an oligopeptide, a polypeptide, an oligonucleotide/oligopeptide hybrid, an oligonucleotide/polypeptide hybrid, an oligonucleotide/oligopeptide hybrid and a polynucleotide/polypeptide hybrid.

In another aspect, the present invention provides methods described above whereby when the NAzyme substrate is subsequently added the cleavage activity is measured using the observed increase in signal or whereby by the NAzyme substrate has a fluorophore on one end and a quencher on the other end so that the fluorescence increases when by cleavage the distance between the fluorophore and quencher increases.

In certain embodiments of the present invention, the target to be analysed is on solution or the target to be analysed is captured on a support on magnetic microbeads.

Some of the methods described above may be embodied for detecting the presence of the disease, a disorder, or biological state, wherein a detectable signal on at least one capture region on the device indicates the presence of the disease, disorder, or biological state in the subject.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### DETAILED DESCRIPTION OF EMBODIMENTS OF THE INVENTION

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof Several documents are cited throughout the text of this specification. Each of the documents herein (including any manufacturer's specifications, instructions etc.) are hereby incorporated by reference; however, there is no admission that any document cited is indeed prior art of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof Thus, the scope of the expression “a device comprising means A and B” should not be limited to the devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description. Other embodiments of the invention will be apparent to those skilled in the art from consideration of the specification and practice of the invention disclosed herein.

It is intended that the specification and examples be considered as exemplary only.

Each and every claim is incorporated into the specification as an embodiment of the present invention. Thus, the claims are part of the description and are a further description and are in addition to the preferred embodiments of the present invention.

Each of the claims set out a particular embodiment of the invention.

The following terms are provided solely to aid in the understanding of the invention.

### DEFINITIONS

A biosensor is an analytical device, used for the detection of an analyte, that combines a biological component with a detector for instance a physicochemical or optical detector.

A CRISPR/Cas guide RNA is the guide RNA (gRNA or sgRNA) of an engineered CRISPR systems contain tracr-RNA and more particularly CRISPR-RNA (crRNA) which is a short synthetic RNA composed of or comprising a scaffold sequence necessary for Cas-binding and a user-defined ˜20 nucleotide sequence that defines the genomic target to be modified. Simply by changing the target sequence present in the crRNA, one can change the genomic target of the Cas protein.

Engineered CRISPR systems contain two components: a guide RNA (gRNA or sgRNA) and a CRISPR-associated endonuclease (Cas protein). The gRNA includes a short synthetic RNA composed of a scaffold sequence necessary for Cas-binding and a user-defined ˜20 nucleotide spacer that defines the genomic target to be modified. Thus, one can change the genomic target of the Cas protein by simply changing the target sequence present in the gRNA.

Nucleic acid enzymes, called NAzymes, or catalytic nucleic acids, are oligonucleotides that are capable of performing a specific chemical reaction, often but not always catalytic, for instance a single-stranded DNA fragment having a catalytic function, capable of specifically recognizing a target mRNA. These may include DNAzymes, RNAzymes, MNAzymes, or any derivative thereof.

We present a method of modifying the guide RNA component of the CRISPR/Cas complex for signal generation. A CRISPR/Cas complex was modified by that NAzyme sequence is included in tracrRNA sequence, resulting in a hybrid RNA-DNA molecule. The hybrid guide RNA successfully formed functional complex with Cas9 and dCas9. The complex with hybrid successfully bind and cleaves target DNA

NAzymes are oligonucleotides with enzymatic capabilities, including RNA and RNA-DNA hybrid cleavage, peroxidase activity [J. Kosman and B. Juskowiak, Anal. Chim. Acta, vol. 707, no. 1, pp. 7-17, 2011], Friedel-Crafts reaction [A. J. Boersma, et al. Angew. Chemie Int. Ed., vol. 48, no. 18, pp. 3346-3348, 2009], and porphyrin metalation [Y. Li and D. Sen, Nat. Struct. Biol., vol. 3, no. 9, pp. 743-747, 1996]. RNA/RNA-DNA hybrid cleaving NAzyme comprises of a catalytic core flanked by two substrate binding arms involved in hybridization with the specific substrate by virtue of Watson-Crick base pairing both present on the uncleaved substrate, such as the 10-23 and 8-17 core NAzymes.

MNAzymes are derived from their parent DNAzymes via division of the catalytic core into two halves, and addition of two binding arms to each of the partial catalytic cores. Thus, the MNAzymes assemble in their catalytic form only in the presence of a facilitator oligonucleotide, binding with the target-binding arms of the MNAzyme and enabling cleavage of substrate, binding to the other two arms [E. Mokany, S. M. et al. Journal of the American Chemical Society, vol. 132, no. 3. pp. 1051-1059, Jan-2010]. MNAzymes only form catalytic core in the presence of an assembly facilitator (dashed strand in FIG. 6B).

NAzymes have been used for sensing related applications, albeit requiring one step or another to occur at elevated temperatures [S. Deborggraeve, J. Y. Det al. Chem. Commun., vol. 49, no. 4, pp. 397-399, 2013; S.-F. Torabi et al., Proc. Natl. Acad. Sci., vol. 112, no. 19, pp. 5903-5908, 2015; D. Mazumdar et al., J. Am. Chem. Soc., vol. 131, no. 15, pp. 5506-5515, 2009; W. Zhou, et al ACS Sensors, vol. 1, no. 5, pp. 600-606, 2016 and R. Saran and J. Liu, Inorg. Chem. Front., vol. 3, no. 4, pp. 494-501, 2016].

### EXAMPLES

### Example 1 Guide RNA Activity With Cas9

FIG. 2 shows the in vitro cleavage assay performed with the original (sgRNA) and engineered guide RNA (hybrid) in the presence of both Cas9 and the non-cleaving dCas9.

Column 8 shows the uncleaved target DNA in nuclease-free water (NF water), and columns 4 & 7 show the target DNA incubated only with sgRNA or hybrid, respectively. The cleavage pattern with sgRNA and hybrid in the presence of Cas9 (columns 2 & 5 respectively) is similar, demonstrating no loss of complex cleavage activity due to engineering of the guide RNA.

The nucleic acid pattern with sgRNA and hybrid in the presence of dCas9 (column 3 & 6 respectively) is similar as well, and shows no cleavage or degradation of target. The cleavage activity is integral to the Cas9 protein, and not dCas9 protein. The results also demonstrate the finding that hybrid RNA does not interfere with the cleavage of Cas9. This is important since DNAzyme itself also has nucleic acid cleavage activity, and any cross-reactivity would undermine the efficacy of the system.

### Example 3 Buffer Optimization for Guide RNA Activity

The buffer conditions required for NAzyme activity are different from those required for CRISPR/Cas recognition and cleavage activity. We assessed the activity of CRISPR/Cas complex using the sgRNA and the hybrid in a panel of buffers to find the optimal one for both functions—CRISPR/Cas recognition and NAzyme activity. It was found that the CRISPR/Cas system functions in a similar manner in all buffers, with both sgRNA and hybrid. It was also demonstrated that the complex is active and functional in the optimal buffer for NAzyme activity as well (Buffer D)

### Example 4 NAzyme Activity of Hybrid RNA

The tracrRNA component includes the NAzyme sequence. We assessed the NAzyme mediated signal generation activity of this hybrid in the presence of NAzyme substrate. The substrate has a fluorophore on one end and a quencher on the other end. Due to NAzyme mediated cleavage of the substrate, the distance between the fluorophore and quencher increases, resulting in increase in fluorescence. The non-cleavable substrate cannot undergo cleavage and hence does not demonstrate the same increase in presence of NAzyme.

FIG. 4 shows a comparison of the NAzyme mediated cleavage activity of hybrid tracrRNA (where NAzyme sequence is added to the tracr sequence) and a nascent NAzyme.

There is difference in the signal generated between nascent NAzyme and hybride, an expected observation due to the steric hindrance posed by accompanying tracr sequences. There is significant increase in signal from hybrid compared with the non-cleavable control, demonstrating the hybrid can be used for NAzyme mediated signal generation.

### Example 5 Application of Engineered System

i. Biosensing utilizing the target recognition ability of CRISPR part of the complex, and signal generation ability of the NAzyme. The schematic in FIG. 5 shows the concept of the bioassay, and the results from the first assessment.

Engineered CRISPR system binds with target DNA functionalized on magnetic microbeads.

NAzyme cleavage dependent increase in fluorescence is observed upon binding with the target DNA.

The signal with target DNA is significantly higher than the background fluorescence and the signal due to non-specific binding.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a schematic view showing wldtype CRISPR/Cas complex (A) and adapted CRISPR/Cas complex (B) that bind target DNA based on base-complementarity with the crRNA and presence of the specific PAM site. Successful binding triggers Cas protein to cleave the target DNA at specified position. Wildtype complex (A) uses two separate RNA molecules; crRNA and tracrRNA. The adapted complex (B) has a linker loop connecting the two RNA molecules, resulting in a single guide RNA (sgRNA).

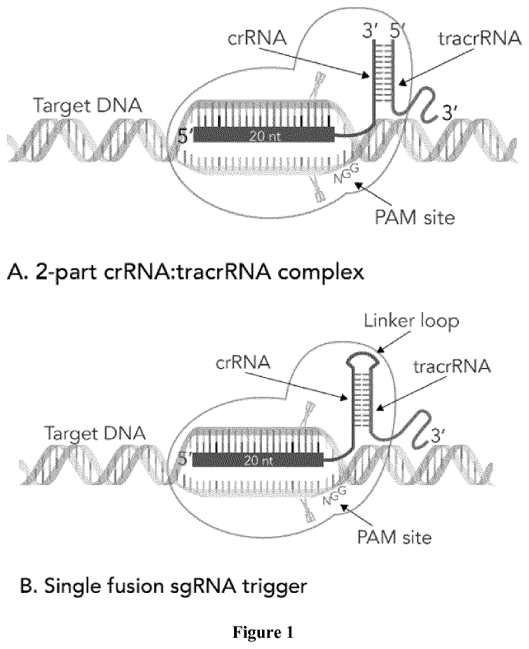

FIG. 2 is photo of an in vitro cleavage assay with original guide RNA (sgRNA) and the engineered RNA-DNA hybrid guide RNA (hybrid) in the presence of Cas9 and dCas9. DNA ladder (1), sgRNA with target and Cas9 (2), sgRNA with target and dCas9 (3), sgRNA in NF water (4), hybrid RNA with target and Cas9 (5), hybrid RNA with target and dCas9 (6), hybrid RNA in NF water (7), and target DNA in NF water (8) are shown. The cleavage activity with both guide RNAs is similar (2&5).

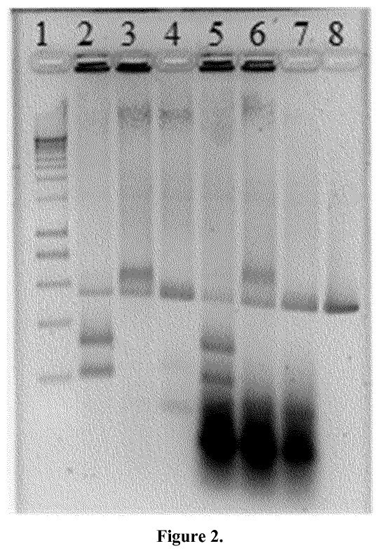

FIG. 3 is a photo demonstrating the activity of both guide RNAs in presence of target DNA and Cas9 in different buffers. The system is demonstrated to be robust enough to function well in all the tested buffers. Buffer D is the optimal buffer for DNAzyme activity. The tested buffers A to D are (A) 200 mM HEPES, 1 M NaCl, 50 mM MgCl2, pH 8.3, (B) 200 mM HEPES, 1 M NaCl, 200 mM MgCl2, 1 mM EDTA, pH 6.5, (C) 200 mM HEPES, 1 M KCl, 50 mM MgCl2, 1 mM EDTA, pH 6.5, (D) 100 mM Tris-HCl, 500 mM KCl, 200 mM MgCl2, pH 8.3

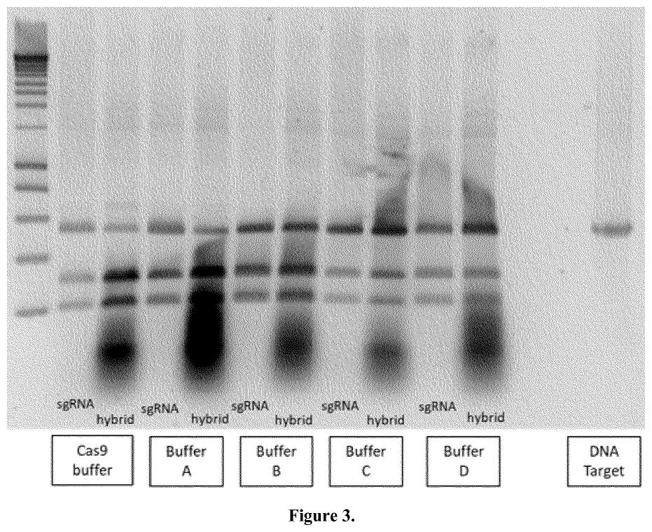

FIG. 4 is a graph demonstrating the NAzyme mediated signal generation of the engineered hybrid tracrRNA: the hybrid with cleavable substrate, the hybrid with non-cleavable substrate, NAzyme with cleavable substrate and NAzyme with cleavable substrate. The activity of original NAzyme is also shown for comparison.

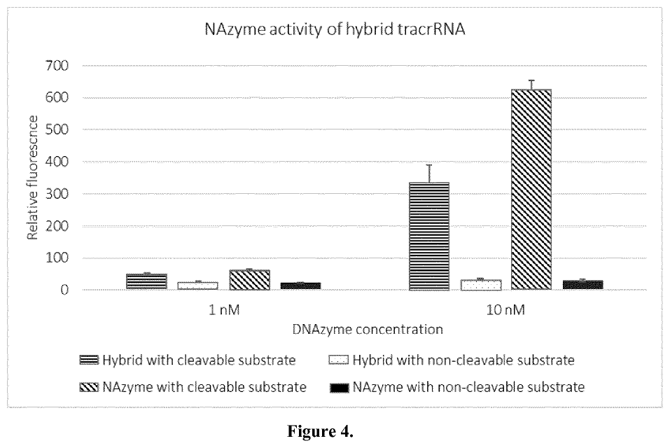

FIG. 5 is schematic displaying of the biosensing application of the engineered CRISPR/Cas system, along with results from the assay. (A) The hybrid tracrRNA (with the NAzyme sequence), crRNA, and dCas9 is incubated together for complex formation. (B) The target DNA is captured on magnetic microbeads (MM), and incubated with the assembled CRISPR/dCas9 complex. The substrate for NAzyme is subsequently added, and cleavage activity measured using the observed increase in fluorescence. (C) The results from the pilot test show a significant increase in fluorescence in the presence of target compared with the fluorescence without target.

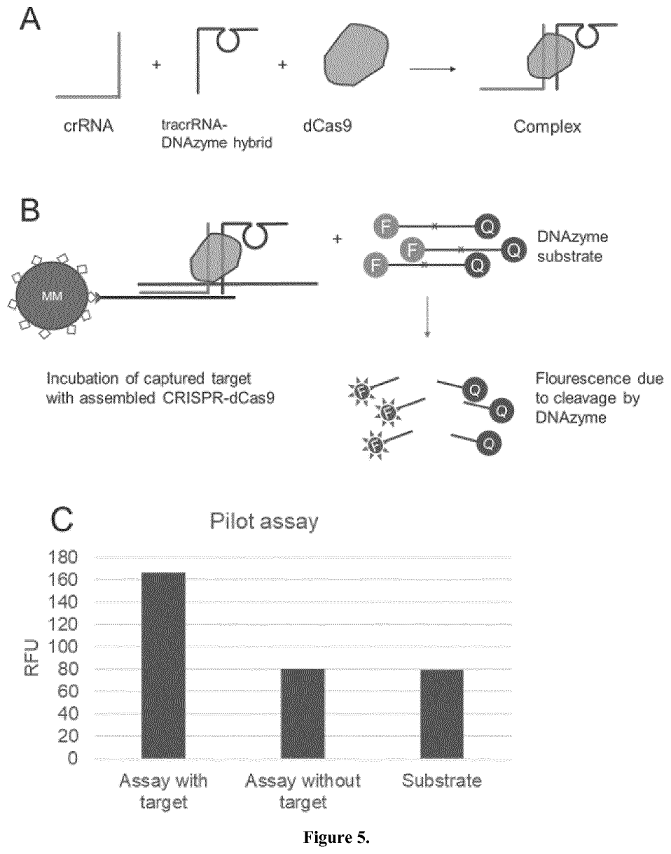

FIG. 6 is a schematic demonstrating DNAzymes (A) and MNAzymes (B) binding cleavable substrate via binding arms, resulting in catalytic cleavage, giving rise to increase in fluorescence (because of separating quencher (Q) from the fluorescent group (F).

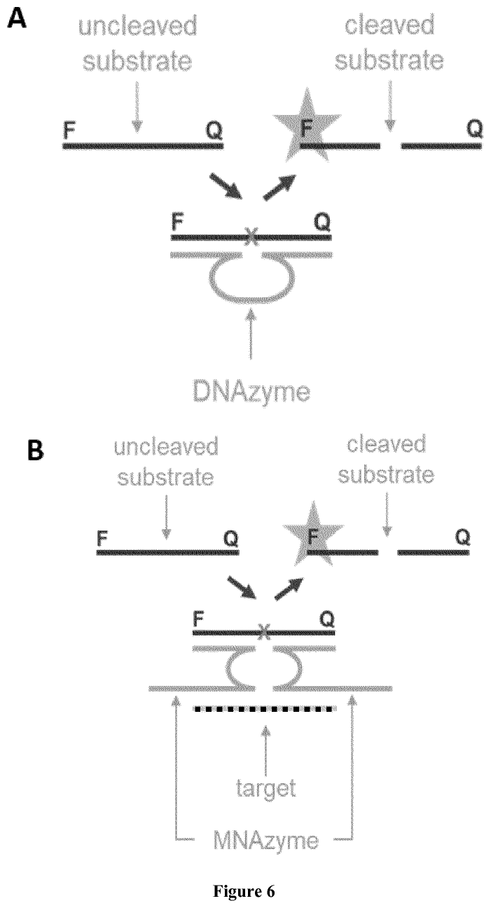

Particular and preferred aspects of the invention are set out in the accompanying independent and dependent claims. Features from the dependent claims may be combined with features of the independent claims and with features of other dependent claims as appropriate and not merely as explicitly set out in the claims.

“Ambient stable ” is stable at ambient condition or in an ambient environment.

Thus, the claims following the detailed description are hereby expressly incorporated into this detailed description, with each claim standing on its own as a separate embodiment of this invention.

1. (canceled)

2. A biosensor comprising a transducer element associated with an engineered CRISPR system comprising a guide RNA-NAzyme hybrid.

3. The biosensor according to claim 2, wherein the engineered CRISPR system is an engineered CRISPR/Cas comprising a tracrRNA-NAzyme hybrid either in a Cas-crRNA-tracrRNA complex or in a dCas-crRNA-tracrRNA complex.

4. The biosensor according to claim 2, wherein the engineered CRISPR system is an engineered CRISPR/Cas comprising a tracrRNA-NAzyme hybrid comprised either in a Cas9-crRNA-tracrRNA complex or in a dCas9-crRNA-tracrRNA complex.

5. The biosensor according to claim 2, wherein the guide RNA-NAzyme hybrid comprises a tracrRNA-NAzyme hybrid having a catalytic motif, and wherein the catalytic motif of the tracrRNA-NAzyme hybrid is a 10-23 NAzymes motif or a 8-17 NAzymes motif.

6. The biosensor according to claim 2, wherein the transducer element is a physicoelectrical transducer.

7. The biosensor according to claim 2, wherein the transducer element is a physicochemical transducer.

8. The biosensor according to claim 2, wherein the transducer element is optochemical transducer.

9. The biosensor according to claim 2, wherein the biosensor is targeted to a target selected from the group consisting of a nucleic acid, a protein/peptide, lipid, polysaccharide, a cell surface, an oligonucleotide, a polynucleotide, an oligopeptide, a polypeptide, an oligonucleotide/oligopeptide hybrid, an oligonucleotide/polypeptide hybrid, an oligonucleotide/oligopeptide hybrid, and a polynucleotide/polypeptide hybrid.

10. The biosensor according to claim 2, adapted to transform the binding and catalytic effect of the engineered CRISPR system into a detectable/measureable electrical indicator.

11. The biosensor according to claim 2, further comprising a reaction portion that is connectable to the transducer element, the biosensor being incorporated into a biosensor system that comprises a measuring instrument that measures biological information on an analyte supplied to the biosensor.

12. The biosensor according to claim 2, wherein the biosensor is disposed on a biosensor chip.

13. The biosensor according to claim 2, wherein the biosensor is disposed on a biosensor chip and is connected with a measuring instrument that measures biological information on a biological material supplied to the biosensor chip.

14-16. (canceled)

17. A sensor kit comprising:
a hybrid tracrRNA-NAzyme in a complex with Cas-crRNA; or
a hybrid tracrRNA-NAzyme in a complex with Cas9-crRNA; or
a hybrid tracrRNA-NAzyme in a complex with dCas-crRNA; or
a hybrid tracrRNA-NAzyme in a complex with dCas-crRNA and further including a substrate for NAzyme.

18-19. (canceled)

20. The biosensor according to claim 2, wherein the NAzyme of the guide RNA-NAzyme hybrid is selected from the group consisting of a DNAzyme, RNAzyme, DNAzyme-RNAzyme hybrid, a multi-component deoxyribozyme (MNAzyme), and any combination thereof.

21. A method for analyzing a target analyte in a biological sample, the method comprising:
(a) contacting a biosensor according to claim 2 with a biological sample for a time sufficient to form a target-analyte/ligand complex by a complex reaction;
(b) converting the complex reaction into a measurable signal; and
(c) transducing the measurable signal into a readable result.

22. The method according to claim 21, wherein the biosensor comprises an engineered CRISPR system with a NAzyme substrate that has a fluorophore on one end thereof and a quencher on the other end thereof so that the fluorescence increases when by cleavage the distance between the fluorophore and quencher increases.

23. (canceled)

24. The method of claim 21, further comprising:
probing the target analyte with a tracrRNA-NAzyme Cas-crRNA complex or with a tracrRNA-NAzyme Cas9-crRNA or with a tracrRNA-NAzyme dCas-crRNA or with a tracrRNA-NAzyme dCas9-crRNA complex; and
adding a labelled NAzyme substrate for the NAzyme in the complex.

25. The method of claim 21, wherein the target analyte is at least one selected from the group consisting of RNA, cDNA, and genomic DNA, the cDNA being obtained from RNA by reverse transcription.

26. The method of claim 21, wherein the target analyte is selected from the group consisting of a nucleic acid, a protein/peptide, lipid, polysaccharide, a cell surface, an oligonucleotide, a polynucleotide, an oligopeptide, a polypeptide, an oligonucleotide/oligopeptide hybrid, an oligonucleotide/polypeptide hybrid, an oligonucleotide/oligopeptide hybrid, and a polynucleotide/polypeptide hybrid.

27-30. (canceled)

31. The method of claim 21, wherein the target analyte is captured on a support or on magnetic microbeads.

32-33. (canceled)
